(19) United States
(12) Patent Application Publication
(10) Pub. No.: US 2013/0289470 A1
(43) Pub. Date: Oct. 31, 2013

(54) KIT FOR THE TREATMENT OF ENVENOMATION

(71) Applicant: Kamal Cherif Zahar, Paris (FR)

(72) Inventor: Kamal Cherif Zahar, Paris (FR)

(21) Appl. No.: 13/996,664

(22) PCT Filed: Dec. 20, 2011
(86) PCT No.: PCT/FR11/53068

§ 371 Date: Jun. 21, 2013

(30) Foreign Application Priority Data
Dec. 24, 2010 (FR) 10 61258

(51) Int. Cl. A61F 17/00

(52) U.S. Cl. CPC A61F 17/00
USPC 604/22

(57) ABSTRACT

A kit for the treatment of envenomation, includes: a syringe with needle, filled with a liquid for local anaesthesia and vasoconstriction, a bistoury having a blade with an incision depth limiter, and a syringe with needle, filled with a washing liquid including the same compounds as those of the syringe with the liquid for local anaesthesia and vasoconstriction, but at a lower concentration. Such a kit permits local emergency extraction of the venom from a bite or sting inoculated by a venomous animal.

The present invention is related to a kit for the urgent treatment of envenomation, in particular a kit aimed at extracting locally venom from wound, bites and stings.

Envenomation is a major problem of public health. The number of snake bites in the world would amount annually to more than 5 million and these would provoke 20 000 to 100 000 death. Besides, it counts for more than 1.2 million of scorpion stings per year, causing around 3000 dead, young children mostly. Insects stings and marine animals are also the cause of accidents needing medical staff care.

The recommendation concerning the primary care in case of sting or bite by a venomous animal are unanimous to advise not to do anything locally on the point of sting or bite, to wait for the appearance of delayed general symptoms from one to two hours and to decide or not an injection of anti-venom serum while treating them symptomatically as and when they appear.

Besides, the inefficiency of the anti-venom serum is disputed since some time while its iatrogenic risks are put forward. In addition, these arrangements require the rapid presence of medical personnel by the victim, which is not always possible.

Now, if a local emergency treatment could be realized immediately after the sting or the bite to remove the venom before it affects and/or it spreads into the organism, and provoke the serious general symptoms that can lead to death, this would enable to limit the importance of the local and general symptoms of envenomation and to reduce considerably the number of mortality associated to the inoculation by venomous animals.

Currently, there exist no efficient solution and adapted to local emergency treatment of venomous stings or bites.

A lot of devices have yet been developed in particular in the form of venom pumps, such as the Aspivenin®, mini pump which is composed of a double room with a block of piston at the end of the race which provokes an important and permanent depression of around 800 milibars. Other embodiments have also been described, such as in the application FR-2739779 or in the application FR-2562423.

However, pursuant to the whole studies and medical and toxicological reports established since a dozen years on this subject, l'Aspivenin® and other venom pumps have no effect and have no point, in particular in the case of snake bites or arachnids stings, because they cannot suck up the venom across the dermis.

This is why the objective of the present invention is to overcome the drawbacks of the previous devices, and to propose a simple solution of use and easily transportable, enabling to treat efficiently an envenomation, by extracting locally the venom and by attempting to neutralize locally what would not have been able to be extracted at the level of the sting or the bite.

To this end, the invention aims at being a kit for the treatment of envenomation, including at least:
a syringe with needle, filled with a liquid for local anesthesia and vasoconstriction,
a bistoury having a blade with an incision depth limiter, and
a syringe with needle, filled with a washing and venom neutralizer liquid.

Advantageously, this kit is simple to manufacture and to use and enables the implementation, immediately after inoculation, of a method enabling to get out the venom of the organism, before it is spread by lymphatic way and to act at distance away, avoiding thereby the appearance of serious symptoms that might be lethal.

The presence of pre-filled syringes and of a bistoury having a blade with an incision depth limiter with no danger, enables an immediate use on the mere site of inoculation, including by a person without experience non belonging to the medical community.

Other characteristics and advantages will come out from the description which will follow the invention, description which is provided as an example only, with regard to the drawings attached on which:

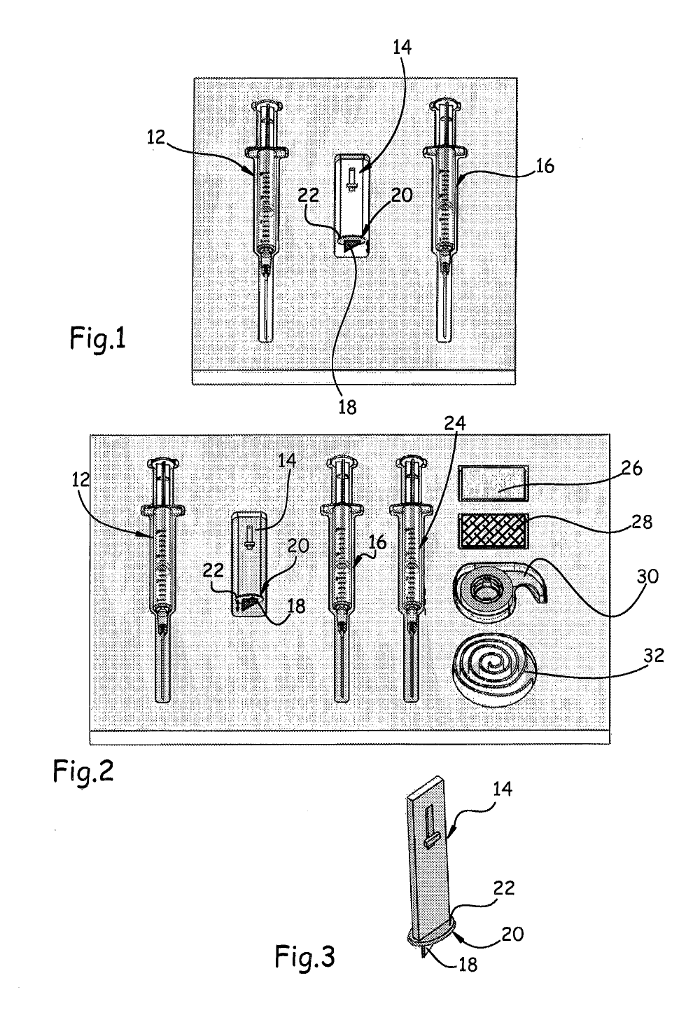

FIG. 1 represents a kit 10 of treatment of envenomation pursuant to the invention,

FIG. 2 represents an embodiment of a kit 10 of treatment of envenomation pursuant to the invention,

FIG. 3 represents a figure of the bistoury 14 of kit 10 of treatment of envenomation pursuant to the invention.

As represented in FIG. 1, the kit 10 of treatment of envenomation pursuant to the invention, includes at least:
a syringe 12 with needle, filled with a liquid for local anesthesia and vasoconstriction,
a bistoury 14 having a blade with an incision depth limiter, and
a syringe 16 with needle, filled with a washing and venom neutralizer liquid.

By liquid for local anesthesia and vasoconstriction, it shall be understood any composition in liquid form presenting at the same time an anesthetic effect and vasoconstrictor including one or more active ingredients. It may notably be molecules selected among anesthetics such as for example the novocain, the xylocain, the marcaine, and vasoconstrictors such as for example the adrenaline, the noradrenaline, the epinephrine and possibly alkaline substances such as bicarbonate of sodium.

Preferably, the liquid contained in the syringe 12 is a mix of xylocain, adrenaline and bicarbonate of sodium.

As a preferred manner, the concentration of the composition contained in the liquid of syringe 12 is comprised between:
5 to 10 mg/ml for the xylocain,
0.0025 to 0.0050 mg/ml for the adrenaline,
Bicarbonate to reach the pH neutral, calculated in respect to the acidity generated by the quantity of xylocain

For the washing and neutralization liquid of syringe 16, it shall be understood the same composition as those of syringe 12 to a less important concentration by dilution of the salted isotonic serum and the products aimed at neutralizing the venom by digestion or modification of the toxic molecules it contains.

Preferably the liquid contained in syringe 16 may be xylocain to a concentration of 0.8 to 0.10 mg/ml, adrenaline to a concentration of 0.001 to 0.002 mg/ml and bicarbonate of sodium at 2.8 mg/ml.

It is also possible to add substances which are able to neutralize the venom in order to make inefficient or with limited effects the sole part of the venom not extracted as it will be later explained.

It may be components such as anti-venom serums but a much lower doses.

The bistoury 14 is constituted of, in a very particular mode, non limitative, a blade 18 with an incision depth limiter 20 in the form of a wing 22 coming in contact with the skin. The blade 18 is retractable in order to deploy it only at the time of utilization. This baled 18 can only take two positions thanks to an associated slider. This particular configuration with wing 22 prevents all risks of too important penetration of the blade in the skin at the time of incision, and enables an easy utilization and non-dangerous, including by non-professional persons who have never used this type of instrument.

The bistoury may also include simply as limiter 20 of incision depth, a blade of short length sleeve preventing from going more in depth.

Pursuant to another embodiment, the bistoury may include a serrage with two depths depending on the thickness of the skin instead of the sting or the bite.

Nonetheless, this embodiment shall be put in regard with the situation that might be of panic at the time of the realization of the act, limiting the analyzing abilities.

It therefore requires a simple and fast manipulation, a bistoury of two retractable/operational positions is therefore preferable.

Pursuant to an embodiment represented in FIG. 2, the kit 10 may also contain one or more syringe 24. In fact, if the victim is treated only lately for any reason whatsoever and that the venom already spread, it requires a more wider treatment with a volume of liquid much more important and injections more distanced and more deep of the point of envenomation.

In that case, it requires more liquid quantities but also to a lesser concentration. The kit 10 pursuant to the invention may also contain:
One or more compresses 26 soaked of antiseptic aimed at sterilizing the wound, and/or
One or more dry compresses 28, an adhesive 30 and/or a tire 32 aimed at realizing a band-aid at the level of the wound after treatment.

It may also possibly contain surgery gloves. All the content of the kit is for single use and industrially sterilized.

Of course, the kit contains an instruction notice explaining the different steps to be implemented to extract the venom of the bite or sting with the help of the different elements it contains.

The kit pursuant to the invention permits the emergency treatment of envenomation.

The method of use of the kit consists in opening it, then in a first phase to jab with syringe 12 in a healthy skin at a small distance of 1 to 3 cm of the point of envenomation, and to inject the liquid it contains in order to obtain a local anesthesia, calming the pain and agitation of the victim. This injection also provokes a vasoconstriction, which reduced the blood circulation locally, also reducing the risks of spread of the venom.

The small molecules of venom spread in a limited manner across the capillary face. The vasoconstriction will also reduce the bleeding occasioned by the incision to come.

Once the zone is treated as such, it is required to incise the skin at the point of envenomation with the blade 18 of bistoury 14 of limited depth incision.

This incision is followed by the injection of the content of syringe 16, at a distance of 3 to 5 cm of the point of envenomation and the incision, which provokes a pressure in the intercellular environment, pressure which makes the venom circulate from inside to outside through the incision. The venom extracts itself from the inoculated zone.

This second syringe essentially aims at washing and neutralizing if possible the fraction of the venom which would not have been extracted thanks to the components of neutralization of said venom.

In the case of the embodiment represented in FIG. 3, the treatment process includes the following steps:
Opening of the kit,
Affixing of the or the compress (es) 26 with antiseptic on the skin, around the bite or sting,
Injection of the content of syringe 12 in at least one point located close to the point of envenomation,
Incision of the skin at the point of envenomation with the blade 18 of bistoury 14,
Injection of the content of syringe 16, in at least a more distant than the at least point of injection of syringe 12
Realization of a band-aid with compresses 28 and the adhesive 30 or the band-aid 32.

It has to be noted that if the kit does not contain surgery gloves, it is possible to realize all the steps directly hand-to-hand, with hand washed or not.

The kit of treatment of envenomation pursuant to the invention, transportable and easy to use, enables an immediate implementation of the treatment at the mere place of the incident, avoiding the spread of substances having general noxious effects by lymphatic ways. The time of intervention is generally comprises between 10 and 20 minutes. It therefore enables to save lives in the most serious cases, or to mitigate the symptoms in the more benign cases.

Advantageously, the treatment with the help of the kit pursuant to the invention does not require any system of sucking. It is the pressure provoked in the intercellular environment by the injection of liquid which makes circulate the venom towards the exterior.

In that case, and after the implementation of the process pursuant to the invention, in addition but in a non-necessary manner, a suck-up can be applied efficiently because the skin is open, leaving the venom pass, and the pressure within the interstitial tissue favor the exit of the or the liquid (s) injected including the venom.

In addition, the products used are without danger, commonly used in all surgery services, both by the person who uses it and the person inoculated who is subject to the treatment.

It is particularly adapted to snake bites and scorpion stings, but may also be used in case of envenomation by inoculation.

1. Kit for the treatment of envenomation comprising:
a first syringe with needle, filled with a liquid for local anesthesia and vasoconstriction,
a bistoury having a blade with an incision depth limiter, and
a second syringe with needle, filled with a washing liquid including the same compounds as those of the first syringe, but at a lower concentration.

2. Kit for the treatment of envenomation according to claim 1, wherein the liquid contained in the first and second syringes includes a component of neutralization of the venom.

3. Kit for the treatment of envenomation according to claim 1, wherein the liquid contained in the first syringe includes an anesthetic selected among Novocain, xylocain, macaine and a vasoconstrictor selected among adrenaline or noradrenaline or epinephrine.

4. Kit for the treatment of envenomation according to claim 1, wherein the liquid of the first syringe includes a pH pad to bring the composition close to the neutrality.

5. Kit for the treatment of envenomation according to claim 4, wherein the pH pad is selected among the alkaline substances.

6. Kit for the treatment of envenomation according to claim 1, wherein a concentration of the liquid of the first syringe is of 5 to 10 mg/ml of xylocain, 0.0025 to 0.0050 mg/ml of adrenaline and bicarbonate of sodium to reach the neutrality of the composition.

7. Kit for the treatment of envenomation according to claim 1, wherein a concentration of the liquid of the second syringe is of 0.8 to 10 mg/ml of xylocain, 0.001 to 0.002 mg/ml of adrenaline and bicarbonate of sodium to reach the neutrality of the composition.

8. Kit for the treatment of envenomation according claim 1, further comprising
one or several other syringes with needle, filled with a liquid having a similar composition as the one of the second syringe, and/or
one or several compresses soaked with antiseptic, and/or
one or several dry compresses, an adhesive, and/or a band-aid.

9. Kit for the treatment of envenomation according to claim 5, wherein the pH pad is constituted of bicarbonate of sodium.
